(19) United States
(12) Patent Application Publication
(10) Pub. No.: US 2026/0201010 A1
(43) Pub. Date: Jul. 16, 2026

(54) INSULIN FRAGMENT TAG AND PHARMACEUTICAL COMPOSITION

(71) Applicant: KOBE GAKUIN EDUCATIONAL FOUNDATION, Kobe-shi, Hyogo (JP)

(72) Inventor: Noriyasu KAMEI, Kobe-shi, Hyogo (JP)

(21) Appl. No.: 19/134,021

(22) PCT Filed: Mar. 8, 2024
(86) PCT No.: PCT/JP2024/009174

§ 371 Date: May 29, 2025

(30) Foreign Application Priority Data
Mar. 9, 2023 (JP) 2023-036542

(51) Int. Cl. C07K 14/62; A61K 47/42; A61P 25/00

(52) U.S. Cl. CPC C07K 14/62; A61K 47/42; A61P 25/00

(57) ABSTRACT

The present invention addresses the problem of providing a polypeptide tag that is capable of delivering peptides or polypeptides to neurons. The problem is solved by an insulin fragment tag for delivering interested molecules to neurons, the insulin fragment tag comprising a polypeptide including an amino acid sequence that has at least 90% identity with the amino acid sequence of at least one chain selected from an insulin A-chain and an insulin B-chain, and the insulin fragment tag having binding capacity insulin receptors.

### TECHNICAL FIELD

Disclosed herein are an insulin fragment tag, a pharmaceutical composition, a polynucleotide encoding the insulin fragment tag, a polynucleotide encoding a peptide or polypeptide having pharmacological activity to which the insulin fragment tag is added, a vector containing said polynucleotide, and a cell into which said vector is introduced.

### BACKGROUND ART

Insulin is produced and secreted by a pancreas and plays a role in regulating blood glucose throughout a body. On the other hand, it has recently been reported that insulin has a function in a brain that promotes memory and learning. In past studies, it has also been shown that efficient delivery of the insulin to the brain via nasal administration leads to improvement of the memory in mouse models of dementia (Non-Patent Literature 1, Patent Literature 1). From this perspective, the insulin produced in periphery has attracted attention in the past as a bioactive substance capable of breaking through Blood-Brain Barrier (BBB), which is a barrier to brain penetration (Non-Patent Literature 2). Based on this, some studies have examined strategies to target insulin receptors on brain capillary endothelial cells, which are components of the BBB, by binding anti-insulin receptor antibodies to drugs to attempt to improve drug permeability (Patent Literature 2). In addition, it has been described that it is possible to deliver a therapeutic payload by administering an antibody that binds to glucose transporter 4 (GLUT4) and the therapeutic payload, and that the activity of the GLUT4 can be enhanced by pretreatment of cells such as a hippocampus, where the GLUT4 is present, with the insulin (Patent Literature 3).

### CITATION LIST

### Patent Literature

[Patent Literature 1] Japanese Translation of PCT International Application Publication No. 2011-504163
[Patent Literature 2] WO2007/044323
[Patent Literature 3] Japanese Translation of PCT International Application Publication No. 2022-540819A

### Non Patent Literature

[Non Patent Literature 1] Pharmaceutics 2021, 13, 1745, 1-15
[Non Patent Literature 2] Pharm Res. 1994 December; 11 (12): 1681-1688.

### SUMMARY OF INVENTION

### Technical Problem

The present invention addresses the problem of providing the polypeptide tag that is capable of delivering the interested molecules to the neurons.

### Solution to Problem

### Item 1.

An insulin fragment tag for delivering interested molecules to neurons, the insulin fragment tag comprising a polypeptide including an amino acid sequence that has at least 90% identity with an amino acid sequence of at least one chain selected from an insulin A-chain and an insulin B-chain, and the insulin fragment tag having binding capacity insulin receptors.

### Item 2.

The insulin fragment tag according to item 1 wherein the insulin fragment tag has no activity as insulin.

### Item 3.

A pharmaceutical composition for treating or preventing a nervous system disease, containing an interested molecule to which the insulin fragment tag added according to item 1.

### Item 4.

The pharmaceutical composition according to item 3, containing a pharmacologically active component in lipid microparticles having the insulin fragment tag on the surface.

### Item 5.

A polynucleotide encoding the insulin fragment tag according to item 1.

### Item 6.

A vector containing the polynucleotide according to item 5.

### Item 7.

A cell into which the vector according to item 6 is introduced.

### Item 8.

A polynucleotide encoding the insulin fragment tag according to item 1 and a peptide or polypeptide that is the interested molecule.

### Item 9.

A vector containing the polynucleotide according to item 8.

### Item 10.

A cell into which the vector according to item 9 is introduced.

### Advantageous Effects of Invention

The polypeptide tag that is capable of delivering the interested molecules to the neurons can be provided.

### BRIEF DESCRIPTION OF DRAWINGS

FIG. 1 shows structures of eight types of expression vectors used in examples.

FIG. 2 shows an image in which localization of nasally administered insulin in a hippocampus is observed.

FIG. 3A shows an image in which expression levels of insulin receptor (Insulin R) in NIH3T3 cells, bEnd.3 cells, C6 cells, and HT22 cells were determined by a Western blot.

FIG. 3B is a graph showing that the expression level of the insulin receptor in FIG. 3A is corrected and quantified by an expression level of β-actin.

FIG. 4 shows results of observation of intracellular localization of the insulin in the four types of cells after the FITC-insulin was taken up by the cells.

FIG. 5A shows results of fluorescence intensity in individual cells taken up the FITC-Insulin compared to untreated cells (negative control) for the NIH3T3 cells, bEnd.3 cells, C6 cells, and HT22 cells.

FIG. 5B shows means and standard deviations of the fluorescence intensities for each cell type in FIG. 5A.

FIG. 6A shows histograms of fluorescence intensity when the FITC-Insulin was taken up by the HT22 cells at 37° C. and fluorescence intensity when the FITC-Insulin was taken up by the HT22 cells at 4° C., as observed by flow cytometry.

FIG. 6B shows means and standard deviations of the fluorescence intensities of the results in FIG. 6A.

FIG. 7 shows images in which accumulation of the insulin in the hippocampus is observed when the insulin is administered nasally.

FIG. 8 shows images in which the accumulation of the insulin in the hippocampus is observed when the insulin is administered nasally.

FIG. 9A shows results of the Western blot to identify a protein purified from Escherichia coli expressing a protein obtained by fusing an insulin fragment to NanoLuc (Nluc). Upper panel shows a result of the Western blot using an antibody recognizing the Nluc, middle panel shows a result of the Western blot using an antibody recognizing insulin A-chain, and lower panel shows a result of the Western blot using an antibody recognizing insulin B-chain.

FIG. 9B shows results of the Western blot to identify a protein purified from Escherichia coli expressing a protein obtained by fusing an insulin fragment to mNeonGreen (mNG). Upper panel shows a result of the Western blot using an antibody recognizing the mNG, middle panel shows a result of the Western blot using an antibody recognizing the insulin A-chain, and lower panel shows a result of the Western blot using an antibody recognizing the insulin B-chain.

FIG. 10A shows insulin activities of polypeptides expressed from each construct.

FIG. 10B shows blood glucose levels in mice administered the polypeptides expressed from each construct.

FIG. 11 shows confocal microscopy images observing results of uptake of insulin fragment-fused mNeonGreen when added to cultured cells.

FIG. 12A shows results of the fluorescence intensity of each cell shown in FIG. 11, analyzed by the flow cytometry.

FIG. 12B shows a graph quantifying the results of FIG. 12A.

FIG. 13A shows results of expression of insulin receptors in primary cultured rat hippocampal neurons as observed by the Western blot.

FIG. 13B shows a graph quantifying the results of FIG. 13A.

FIG. 13C shows confocal microscopy images when the insulin fragment-fused mNeonGreen was added to the cultured cells.

FIG. 14 shows results when the insulin fragment-fused mNeonGreen was administered intracerebroventricularly to a mouse.

FIG. 15 shows a distribution of HtB-mNG in plasma and in each brain tissue when the HtB-mNG was administered nasally.

FIG. 16A shows sequences of the insulin fragments.

FIG. 16B shows respective mean fluorescence intensities and standard deviations in each cell.

FIG. 17 shows results of a Western blotting for extracellular vesicles when a construct in which the insulin fragment was added to PTGFRN was expressed.

FIG. 18 shows results of the Western blotting for the extracellular vesicles when the construct in which the insulin fragment was added to the Lamp2b was expressed.

FIG. 19 shows confocal microscopy images of the extracellular vesicles added to culture supernatants of the C6 cells and HT22 cells.

### DESCRIPTION OF EMBODIMENTS

### 1. Insulin Fragment Tag

One embodiment of the present invention relates to an insulin fragment tag. The insulin fragment tag can be added to an interested molecule thereby enabling delivery of said interested molecule to a neuron.

The neuron is preferably a cell of a central nervous system cell, more preferably a neuron of a dentate gyrus of a hippocampus. The neuron is a cell that expresses an insulin receptor. Here, the neuron does not include a glial cell such as an astrocyte, microglia, or oligodendrocyte, and a cell of a blood-brain barrier.

For insulin, a signal peptide (24 amino acids) and c-peptide (31 amino acids) are cleaved out from preproinsulin (110 amino acids) registered as NCBI Reference Sequence: NP_000198.1 (SEQ ID NO: 1) by processing, and an A-chain and B-chain are then assembled together by a disulfide bond to form mature insulin. In the NP_000198.1, the 25th to 54th amino acid sequence (30 amino acids: FVNQHLCGSHLVEALYLVCGERGFFYTPKT (SEQ ID NO: 3)) is the B-chain, the 57th to 87th amino acid sequence is the c-peptide, and the 90th to 110th amino acid sequence (21 amino acids: GIVEQCCTSICSLYQLENYCN (SEQ ID NO: 2)) is the A-chain.

The insulin fragment tag is not limited as long as it contains a polypeptide including an amino acid sequence that has at least 90% identity with the amino acid sequence of at least one chain selected from the insulin A-chain and the insulin B-chain and it can bind to the insulin receptor. The insulin fragment tag may contain only the A-chain, or a polypeptide having 90% or more identity with the A-chain, or may contain only the B-chain, or a polypeptide having 90% or more identity with the B-chain. It may also be a combination such as, the A-chain (or the polypeptide having 90% or more identity with the A-chain) and the B-chain (or the polypeptide having 90% or more identity with the B-chain), the A-chain (or the polypeptide having 90% or more identity with the A-chain) and the c-peptide (or the polypeptide having 90% or more identity with the c-peptide) and the B-chain (or the polypeptide having 90% or more identity with the B-chain), the A-chain (or the polypeptide having 90% or more identity with the A-chain) and the c-peptide (or the polypeptide having 90% or more identity with the c-peptide), or the c-peptide (or the polypeptide having 90% or more identity with the c-peptide) and the B-chain (or the polypeptide having 90% or more identity with the B-chain).

The identity with the amino acid sequence is preferably 91% or more, 92% or more, 93% or more, 94% or more, 95% or more, 96% or more, 97% or more, 98% or more, 99% or more, or 100%. The amino acid sequence of the non-identical region can include a substitution, deletion, or insertion of an amino acid residue relative to a wild-type amino acid sequence.

In addition, in the present description, amino acid sequences in which amino acids constituting the amino acid sequence represented by the SEQ ID NO: 2 are rearranged (e.g., QLENYCNTSICSLYGIVEQCC: SEQ ID NO: 4, ILQSCNCEYTIQCYVESGNLC: SEQ ID NO: 5) are treated as the same as the amino acid sequence represented by the SEQ ID NO: 2 as long as a polypeptide composed of said amino acid sequence can bind to the insulin receptor. Amino acid sequences in which amino acids constituting the amino acid sequence represented by the SEQ ID NO: 3 are rearranged (e.g., ERGFFYTPKTLVEALYLVCGFVNQHLCGSH: SEQ ID NO: 6, EGYKCPQLTSVHFGLVALFVGERHCFYTLN: SEQ ID NO: 7) are treated as the same as the amino acid sequence represented by the SEQ ID NO: 3 as long as a polypeptide composed of said amino acid sequence can bind to the insulin receptor. The rearrangement can be performed by segmenting the amino acid sequence represented by the SEQ ID NO: 2 into units of 1, 2, 3, 4, 5, 6, 7, 8, 9, 10, or 11 amino acid residues. The rearrangement can be performed by segmenting the amino acid sequence represented by the SEQ ID NO: 2 or SEQ ID NO: 3 into units of 1, 2, 3, 4, 5, 6, 7, 8, 9, 10, 11, 12, 13, 14, or 15 amino acid residues. The number of amino acid residues constituting the unit to be combined may be the same or different. The number of amino acid residues of the polypeptide composed of a rearranged sequence of the amino acid sequence represented by SEQ ID NO: 2 is about 18 to 23, preferably 21. The number of amino acid residues of the polypeptide composed of a rearranged sequence of the amino acid sequence represented by SEQ ID NO: 3 is about 27 to 33, preferably 30.

Binding of the insulin fragment tag to the insulin receptor can be evaluated by bringing cells expressing the insulin receptor, such as NIH3T3 cells and HT22 cells, into contact with a fluorescent dye-labeled insulin in vitro and observing whether the fluorescent dye is taken up by the cells, as described in the examples below. The contact between the cells and the fluorescent dye-labeled insulin is preferably performed at room temperature or above, preferably at 37° C. The uptake of the fluorescent dye can be observed by fluorescence microscopy or by flow cytometry. The insulin receptor is preferably derived from a human.

In addition, the insulin fragment tag itself preferably has no activity as the insulin. The activity as the insulin is intended to as an action of lowering blood glucose level, and 1 unit (1 U) of insulin activity is defined internationally as the smallest amount that can lower blood glucose level to a level that causes a convulsion within 3 hours (blood glucose level: about 45 mg/dL) after a healthy rabbit weighing about 2 kg is fasted for 24 hours and is injected with the insulin. In the present description, having no activity as the insulin is intended that the insulin activity of 1 nM of the insulin fragment tag is less than 1 mU/L as determined by, for example, a human insulin assay kit (Mercodia Human Insulin ELISA Kit: Mercodia, Inc). In said kit, it is specified as 23 U/1 mg of insulin.

The interested molecule is a molecule desired to be delivered to the neuron and to which the insulin fragment tag is to be added. The interested molecule is not limited. For example, the molecule can include peptides, polypeptides, proteins, nucleic acids, lipids, compounds, and the like. Preferably, the interested molecule has pharmacological activity. Alternatively, the interested molecule is bound to a pharmacologically active molecule or constitutes a part of a microparticle containing the pharmacologically active molecule.

In the present description, the peptide has a structure in which 2 to 10 amino acids are bonded together by peptide bonds. Polypeptides have a structure in which 11 or more amino acids are bonded by peptide bonds. The peptide or polypeptide may be linear or cyclic, and may have branched chains. The peptide or polypeptide may also form, in whole or in part, a cyclic structure.

In the present description, the protein is intended to be a state in which the peptide or polypeptide forms a conformation. The conformation can include secondary, tertiary, and quaternary structures. Preferably, a complex structure, i.e., the quaternary structure, of the peptide and/or polypeptide is included. In the protein, it is only necessary that the insulin fragment tag is added to at least one of the peptides and/or polypeptides constituting the protein.

The insulin fragment tag can be added to the peptide, polypeptide, or protein using genetic recombination technology. A peptide, polypeptide, or protein added with the insulin fragment tag can be prepared by gene transfer of a fusion polynucleotide obtained by fusing the nucleotide sequence of a polynucleotide encoding the insulin fragment tag (described below) with the nucleotide sequence of a polynucleotide encoding the peptide and/or polypeptide into a host cell and then expressing the fusion polynucleotide. The host cell can include Escherichia coli, yeast, mammalian cells, insect cells, and avian (preferably chicken) cells. Collection of the peptide, polypeptide, or protein added with the insulin fragment tag from the transfected host cell is known. For example, the fusion polynucleotide may be added with the tag. A nucleotide sequence encoding the tag, such as a histidine tag, FLAG® tag, hemagglutinin (HA) tag, glutathione S-transferase (GST) tag, myc tag, and the like, is added to the nucleotide sequence encoding the peptide or polypeptide added with the insulin fragment tag to be expressed by the host cell as a fusion polypeptide of the insulin fragment tag, the peptide and/or polypeptide and the tag. The expressed fusion polypeptide is purified by a method corresponding to each tag.

When the interested molecule is the peptide or polypeptide, the insulin fragment tag may be added to an N-terminal side or C-terminal side of the peptide or polypeptide. In addition, a linker of about 1 to 10 amino acid residues may be sandwiched between the insulin fragment tag and the peptide or polypeptide.

One embodiment of the present invention relates to the polynucleotide encoding the insulin fragment tag. The polynucleotide is not limited as long as it encodes a nucleotide sequence containing an insulin fragment tag that can bind to a human insulin receptor. For example, when the insulin fragment tag is expressed in Escherichia coli or the like, codons may be optimized to be easily expressed in the Escherichia coli or the like.

The polynucleotide may be single or double stranded. In addition, the polynucleotide may be DNA or RNA.

One embodiment of the present invention relates to a vector containing the polynucleotide. The vector containing the polynucleotide, in other words, is intended that the above polynucleotides are inserted into the vector. The vector may be a cloning vector or an expression vector. In addition, the vector can be selected from plasmid vectors, viral vectors, and transposon vectors according to the host cell.

One embodiment of the present invention relates to a cell into which the vector containing the polynucleotide is introduced. The cell can be the Escherichia coli, yeast, mammalian cells, insect cells, and the like.

When the interested molecule is a nucleic acid, the nucleic acid may be DNA or RNA. The nucleic acid can include the DNA and/or RNA composed of nucleotides having the same structure as natural nucleotides, as well as an artificial nucleic acid composed of nucleotides having a structure different from natural nucleotides. The length of the nucleic acid is not limited. A method for labeling the nucleic acid with the insulin fragment tag can employ a known method. For example, labeling at the 5′ or 3′ end; random labeling, and the like can be included. One example of a method of labeling the nucleic acid with the insulin fragment tag is to bind an avidinylated (or streptavidinylated) insulin fragment tag to a biotinylated nucleic acid. Instead of biotin, a labeling substance such as a fluorescent protein such as FITC may be labeled on the nucleic acid, and then the insulin fragment tag labeled with an antibody that binds to the labeling substance or an antigen-binding fragment thereof may be bound.

### 2. Pharmaceutical Composition

One embodiment of the present invention relates to a pharmaceutical composition containing the interested molecule to which an insulin fragment tag is added described in the above section 1.

For the interested molecule, the description in the above section 1 is hereby incorporated herein. In this embodiment, the interested molecule may have the pharmacological activity to treat or prevent a nervous system disease, or may assist in delivering a component having the pharmacological activity (pharmacologically active component) to the neuron. The pharmaceutical composition is used to treat or prevent the nervous system disease.

The nervous system disease may include neurodegenerative diseases such as dementia, Parkinson's disease, amyotrophic lateral sclerosis, progressive supranuclear palsy, multiple system atrophy, triplet repeat disease and the like, cerebral infarction, or the like.

The dementia includes Alzheimer's disease, cerebrovascular dementia, frontotemporal dementia such as Pick's disease, dementia with Lewy body type, and dementia caused by infectious diseases (such as spirochete infection, HIV infection, prion infection). Mild cognitive impairment such as MCI is also included.

The multiple system atrophy includes striatonigral degeneration, olivopontocerebellar atrophy, Shy-Drager syndrome, and the like. The olivopontocerebellar atrophy is preferred.

The triplet repeat disease includes Huntington's disease, Friedreich's ataxia, spinocerebellar ataxia type 1, and the like.

The cerebral infarction refers to a condition in which the neurons and/or glial cells have died or are likely to die due to ischemia of brain tissue. In addition, the “cerebral infarction” in this aspect shall include cerebral infarction in subacute to chronic phase and prolonged cerebral infarction. The “cerebral infarction” in this aspect is preferably the cerebral infarction in the subacute to chronic phase and the prolonged cerebral infarction.

In this embodiment, the prevention includes inhibiting and/or delaying the onset of a symptom. In addition, the treatment includes relieving and/or disappearing the onset of the symptom.

The pharmaceutical composition can be a formulation for infusion, formulation for nasal administration, and the like. The pharmaceutical composition can contain a carrier appropriate for each dosage form in addition to the interested molecule to which the insulin fragment tag is added described in the above section 1.

When the pharmaceutical composition is the formulation for infusion, it can be prepared by dissolving the interested molecule to which the insulin fragment tag is added described in the above section 1 in an isotonic electrolyte infusion formulation based on a saline, Ringer's solution, or the like.

When the pharmaceutical composition is the formulation for nasal administration, it can be in a dosage form such as liquid, gel, or powder. When the formulation for nasal administration is the liquid, as the carrier, for example, water, ethyl alcohol, macrogol, propylene glycol, ethoxylated isostearyl alcohol, polyoxylated isostearyl alcohol, polyoxyethylene sorbitan fatty acid esters, and the like can be used. In addition, the carrier can contain a pH adjuster such as sodium citrate, sodium acetate, or sodium phosphate; a buffer such as Dibasic Potassium Phosphate, trisodium phosphate, sodium hydrogen phosphate, or sodium citrate; or a stabilizer such as sodium pyrosulfite, EDTA, thioglycolic acid, or thiolactic acid. When the formulation for nasal administration is the gel, as the carrier, for example, cellulose and cellulose derivative such as hydroxypropyl cellulose and hydroxyethyl cellulose, polysaccharide, carbomer, acrylic polymers such as Carbopol®, polyvinyl alcohol and other vinyl polymer, povidone, colloidal silicon dioxide such as AEROSIL® 200 or CAB-O-SIL®, lipophilic silicon dioxide such as AEROSIL® R972, cetyl alcohol, stearic acid, glyceryl behenate, wax, beeswax, petrolatum, lipophilic gum, triglycerides, lanolin, inulin, and the like can be used. When the formulation for nasal administration is the powder, as the carrier, for example, sugars such as mannitol, inositol, maltose, sucrose, lactose, and the like can be used.

A maximum dose per day of the pharmaceutical composition is 100 mg, preferably 50 mg, more preferably 20 mg, more preferably 10 mg, and even more preferably 5 mg, per adult, in terms of the interested molecule. A minimum dose per day is 10 mg, preferably 5 mg, more preferably 2 mg, and even more preferably 1 mg, per adult, in terms of the interested molecule. A range of the dose per day of the interested molecule can be set accordingly based on the above maximum and minimum dose values.

When the interested molecule to which the insulin fragment tag is added described in the above section 1 is administered, it is preferable to administer L-penetratin in combination. A dose of the L-penetratin can be used in a range that exerts a delivery effect of the interested molecule to which the insulin fragment tag is added and does not inhibit the activity of or insolubilize the interested molecule to which the insulin fragment tag is added. For example, the L-penetratin can be administered in the range of 0.1 mM to 3 mM, preferably 0.5 mM to 2 mM per dose.

The pharmaceutical composition may contain the pharmacologically active component in a lipid microparticle. The lipid microparticle has the insulin fragment tag on its surface. That is, the lipid microparticle can bind to the insulin receptor via the insulin fragment tag. The lipid microparticle may also be referred to as a lipid nanoparticle, but its size does not have to be strictly on the order of nanometers. Generally, the lipid microparticles have an average particle size of about 1 nm to 1,000 nm. The average particle size can be measured, for example, by a photon correlation spectroscopy using a submicron particle analyzer N5 (Beckman Coulter, Inc.). As for a structure of a membrane of the lipid microparticle, layers of lipids may be in a one-layer structure or a two-layer structure. The two-layer structure is preferred. The lipid microparticle can include extracellular vesicle, microvesicle, and the like. The lipid microparticle can contain the pharmacologically active component. The insulin fragment tag may be added to the pharmacologically active component itself or to a membrane component composing the lipid microparticle. The membrane component composing the lipid microparticle is composed of lipids. Preferably, a hydrophobic group consisting of a fatty acid and a hydrophilic group containing glycerol and phosphoric acid are contained in one molecule. As the membrane component, phospholipid is more preferable. The insulin fragment tag may be added to the lipids composing the membrane component or to a presenting portion outside of the membrane of the peptide, the polypeptide, or a nucleic acid that are present within the membrane.

The lipid microparticles can be obtained, for example, by expressing the interested molecule to which the insulin fragment tag is added in cells and collecting the lipid microparticles from a culture supernatant of the cells. Alternatively, the lipid microparticles can be produced by incorporating the interested molecule to which the insulin fragment tag is added into the lipid microparticles using Bangham method (lipid thin film method), supercritical carbon dioxide spray method, ultrasonic irradiation method, or the like.

### EXAMPLES

Hereinafter, the present invention will be described in more detail with reference to examples. However, the present invention should not be construed as limited to the examples.

### 1. Materials and Methods

### (1) Cells and Animals

bEnd. 3 cells (mouse brain microvascular endothelial cells: blood-brain barrier cells), C6 cells (astrocytes) and HEK293T (used as extracellular vesicle-producing cells) were purchased from the American Type Culture Collection (ATCC). HT22 cells (neurons) were purchased from Merck Millipore. NIH3T3 cells (fibroblasts) were purchased from RIKEN BioResource Research Center. Six-week-old male ddY mice were purchased from Japan SLC, Inc.

### (2) Preparation and Staining of Tissue Sample

PBS was perfused from a heart, and subsequently 4% paraformaldehyde (PFA) phosphate buffer was perfused. An isolated brain was immersed in 4% PFA solution for 24 hours and in sucrose solution for 48 hours, and then embedded in OCT compound to prepare a frozen brain block. A cryostat was used to prepare 20-μm sections, which were treated with a blocking agent (Nacalai Tesque, Inc., Blocking One Histo) and primary antibodies against insulin and various brain cell marker proteins were added (rabbit anti-insulin (Abcam), rat anti-mouse Nestin (FUJIFILM Wako Pure Chemical Corporation), mouse anti-GFAP (Santa Cruz Biotechnology), mouse anti-MAP2 (BioLegend, Inc.)). After reacting with fluorescence-labeled secondary antibodies, samples for microscopic observation were prepared using DAPI-containing mounting media. Localization of the insulin and various cell markers in a hippocampus and dentate gyrus was observed using a confocal microscope (Olympus Corporation, FV3000).

### (3) Preparation of Insulin Fragment-Fused Polypeptide

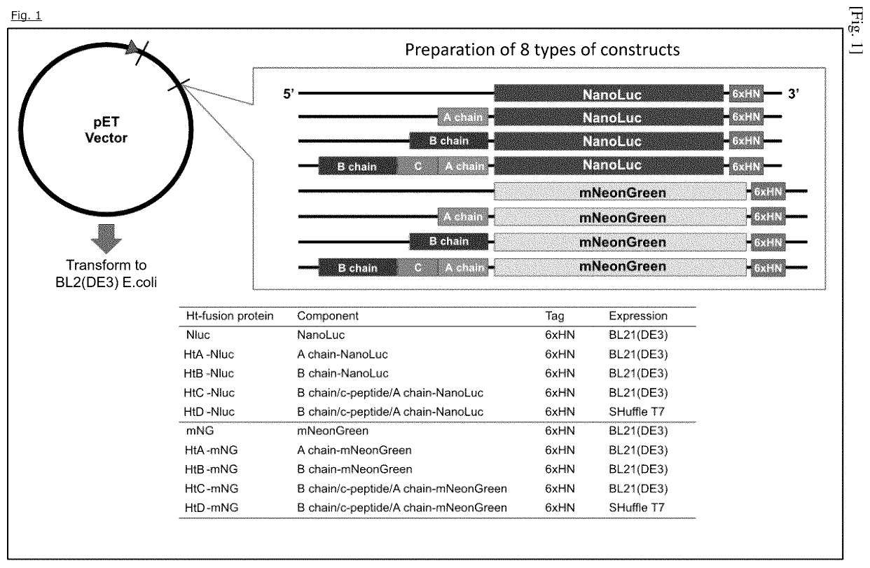

Based on a DNA fragment of whole insulin (synthesized by Integrated DNA Technologies, Inc.), an A-chain (HtA), B-chain (HtB), and B-chain/c-peptide/A-chain fragment (HtC) were prepared by PCR. Primers for the PCR were synthesized by Eurofins. The aforementioned insulin DNA fragment, NanoLuc (Promega Corporation) DNA fragment, and mNeonGreen (Allele Biotechnology) DNA fragment were inserted into pET vectors to prepare eight types of constructs shown in FIG. 1. These were transformed into Escherichia coli (BL21 (DE3) or SHuffle), and then fusion proteins expressed by adding Isopropyl β-D-thiogalactopyranosie (IPTG, FUJIFILM Wako Pure Chemical Corporation) were purified using His60 Ni Gravity Column (Takara Bio Inc.). Concentrations of purified proteins were measured by BCA assay. Insulin fragment-fused NanoLuc was added with a luminescent substrate (Promega Corporation, NanoGlo Luciferase Assay System) and luminescence was measured using a microplate reader. Fluorescence of the insulin fragment-fused mNeonGreen was also measured using the microplate reader.

Insulin activity was measured using a human insulin assay kit (Mercodia Human Insulin ELISA Kit: Mercodia).

### (4) Western Blot

Expression levels of insulin receptors in each type of various cultured cells were compared by a Western blot. NIH3T3 cells, bEnd.3 cells, C6 cells, HT22 cells, and primary cultured rat hippocampal neurons were seeded in 6-well cell culture plates, and one day later, RIPA buffer was added to lyse the cells. A total protein concentration in a sample of lysed cells was measured by the BCA assay, and then 6 μg of the total protein was mixed with sample buffer, heat denatured and used for electrophoresis (SDS-PAGE). After transfer to a PVDF membrane, they were incubated with mouse anti-insulin receptor antibodies (Santa Cruz Biotechnology) and HRP-conjugated goat anti-mouse secondary antibody (Abcam). After reaction with a chemiluminescent reagent (ChemiLumi One Super, Nacalai Tesque, Inc.), the proteins were detected using a ChemiDoc Touch MP (Bio-Rad Laboratories, Inc.).

For identification of the insulin fragment-fused polypeptide by the Western blot, 75 ng of the purified insulin fragment-fused polypeptide was mixed with the sample buffer and used for the electrophoresis under reducing (SDS-PAGE) and non-reducing (Native PAGE) conditions. After transfer to the PVDF membrane, they were reacted with mouse anti-NanoLuc antibody (Promega Corporation), mouse anti-mNeonGreen antibody (Proteintech), rabbit anti-insulin A-chain antibody (Abcam), mouse anti-insulin B-chain antibody (Santa Cruz Biotechnology), and HRP-conjugated goat anti-mouse and anti-rabbit secondary antibodies (Abcam) to detect each protein as described above.

### (5) In Vitro Intracellular Uptake Experiment

Confocal Microscopy: Each type of cells was seeded in a glass-based dish, and after 24 hours, 100 μL of the insulin fragment-fused mNeonGreen (25 μg/mL) was added. It was maintained at 37° C. or 4° C. during incubation. After 110 minutes, Hoechst33342 was added, and after another 10 minutes, solution was removed and washed with glycine HCl buffer or PBS. The cells were not fixed, and the cells were observed as live cells using a confocal microscope (Olympus Corporation, FV3000).

Flow cytometry: Each type of cells was seeded in a 24-well culture plate, and after 24 hours, 500 μL of the insulin fragment-fused mNeonGreen (10 or 25 μg/mL) was added. After 120 minutes, the plate was washed with glycine HCl buffer or PBS, and 200 μL of 0.25% trypsin solution was added to detach the cells. After adding 800 μL of PBS, the cells were passed through a cell strainer and used for flow cytometry (BD, FACSCanto).

### (6) In Vivo Mouse Nasal Administration Experiment

After a ddY mouse was anesthetized, a total of 10 μL of drug solution was administered in 5 μL each into right and left nasal cavities. The insulin, insulin fragment-fused NanoLuc, and L-penetratin were administered at concentrations of 30 IU/mL, 250 μg/mL, and 2 mM, respectively. Distribution of the insulin in a brain was evaluated by immunostaining of mouse brain tissue. 45 minutes after administration of a mixed solution of the insulin and L-penetratin, PBS was perfused from a heart, and subsequently 4% paraformaldehyde (PFA) phosphate buffer was perfused. A tissue sample was prepared and stained according to the method described in the above section (2), and localization of the insulin and various cell markers in the hippocampus and dentate gyrus was observed using the confocal microscope (Olympus Corporation, FV3000). Brain penetration of the insulin fragment-fused NanoLuc was evaluated by mixing supernatants from homogenates of each brain region isolated 15, 30, and 60 minutes after the administration with a luminescent substrate reagent (Promega Corporation, NanoGlo Luciferase Assay System) and measuring emission intensity.

### (7) In Vivo Mouse Intracerebroventricular Administration Experiment

The ddY mouse was anesthetized, then punctured at a position 1 mm to the right of a bregma, and FITC-labeled insulin (150 or 750 μg/mL; 50 or 250 μg/kg as a dose) or insulin fragment-fused mNeonGreen (500 μg/mL; 166.7 μg/kg as a dose) was perfused at a rate of 1 μL/min for 10 minutes. 15 or 30 minutes after administration, the PBS was perfused from the heart, and subsequently the 4% paraformaldehyde (PFA) phosphate buffer was perfused. An isolated brain was immersed in 4% PFA solution for 24 hours and in sucrose solution for 48 hours, and then embedded in OCT compound to prepare a frozen brain block. The cryostat was used to prepare 20-μm sections, and samples for microscopic observation were prepared using the DAPI-containing mounting media. Distribution of the FITC-insulin and insulin fragment-fused mNeonGreen in the hippocampus and dentate gyrus was observed using the confocal microscope (Olympus Corporation, FV3000).

### (8) In Vivo Mouse Subcutaneous Administration Experiment

After the ddY mouse was anesthetized, human insulin or the insulin fragment-fused mNG was administered subcutaneously. The dose of the human insulin was 1 IU/kg (insulin 1.88 μM at 100 μL/30 g body weight was administered), and the insulin fragment-fused mNG was adjusted to the same molar concentration and administered subcutaneously. Thereafter, a blood glucose level was measured with a glucose meter over time. The blood glucose level immediately before the administration was set to 100%, and a change over time in the blood glucose level after the administration was indicated.

### (9) Methods for Functionalization and Collection of EV

The insulin fragment was modified into an extracellular vesicle (EV) as a carrier for carrying a nucleic acid drug. In addition, for quantitative and qualitative analysis of the EVs that are accumulated in or taken up by cells, mNG was included inside the EVs. In order to present the insulin on the EV surface, a construct was prepared to express a protein in which the insulin fragment was fused to an amino terminus of Lamp2b or PTGFRN, which are EV scaffold proteins. In order to include the mNG inside the EV, a construct was prepared to express a protein in which the mNG was fused to a carboxy-terminus of CD63, Lamp2b, or PTGFRN, which are the EV scaffold proteins.

HEK293T cells were seeded in a 15 cm culture plate, and constructs for expression of an insulin fragment-fused EV scaffold protein and mNG-fused EV scaffold protein were added to the cells with polyethyleneimine (gene transfer agent) after 24 hours. After 4 to 6 hours, medium was replaced with serum-free medium, and a culture supernatant was collected after 48 hours. After cell fragments were removed from the culture supernatant by centrifugation, and a sample concentrated using a spin filter was purified by size exclusion chromatography. After further concentration, particle concentration and size were measured by nanoparticle tracking analysis, and fluorescence intensity was measured using a fluorescence plate reader. The amount of the EV added to the cells was adjusted to achieve the same fluorescence intensity among groups.

### 2. Results

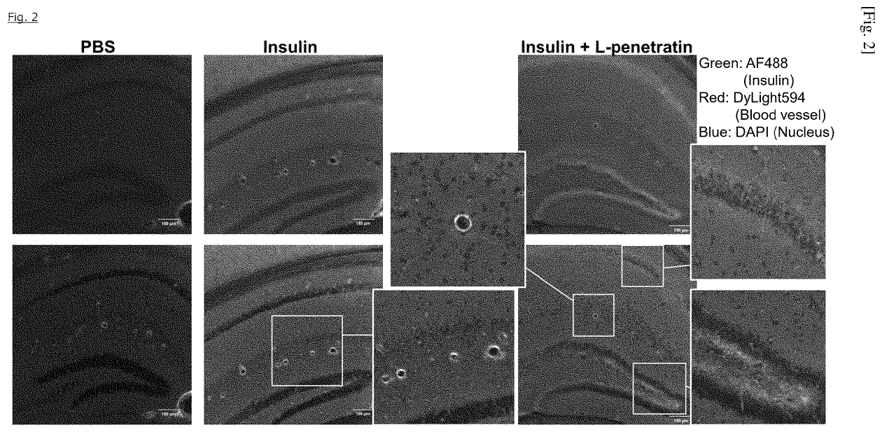

FIG. 2 shows an image in which localization of the nasally administered insulin in the hippocampus is observed. The insulin is stained green with Alexa Fluor® 488 (hereinafter also referred to as “AF488”). Blood vessel is stained red with DyLight™ (trademark) 594. Nucleus is stained light blue with DAPI. The insulin was strongly stained in the dentate gyrus.

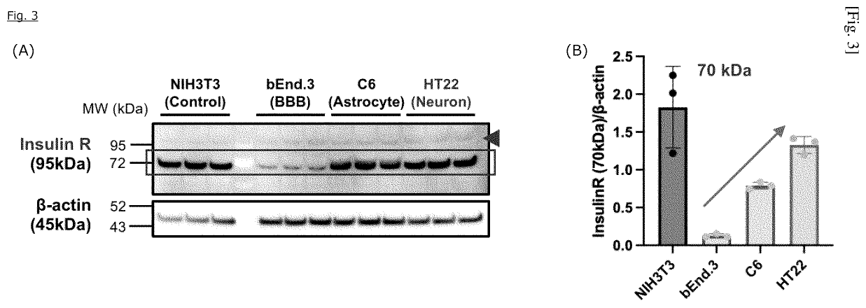

FIG. 3 shows results of observing expression levels of the insulin receptor (Insulin R) in the NIH3T3 cells, bEnd.3 cells, C6 cells, and HT22 cells. FIG. 3A shows an image of the Western blot, and FIG. 3B is a graph showing that the expression level of the insulin receptor is corrected and quantified by an expression level of B-actin. HT22 cells, which are neurons, showed higher expression level of the insulin receptor than that of the bEnd.3 cells and C6 cells, which are cells of other brain tissues.

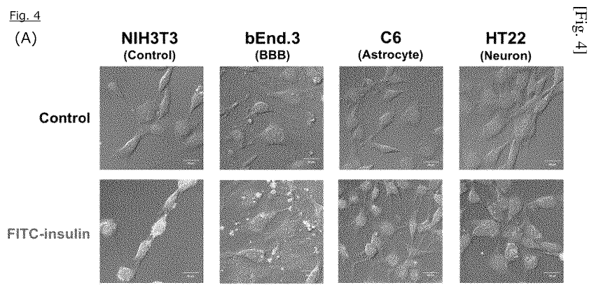

FIG. 4 shows results of fluorescence observation of insulin localization in the NIH3T3 cells, bEnd.3 cells, C6 cells, and HT22 cells. By the confocal microscopy, images of the FITC-labeled insulin (FITC-Insulin) accumulating inside the cells, especially around the nuclei, was observed. In addition, FITC signals inside the cells were stronger in the NIH3T3 cells, C6 cells, and HT22 cells, which showed higher expression level of the insulin receptor, while the FITC signals inside the cells were weaker in the bEnd.3 cells, which showed lower expression level of the insulin receptor. These results suggest that the efficiency in intracellular uptake of the insulin depends on the expression level of the insulin receptor.

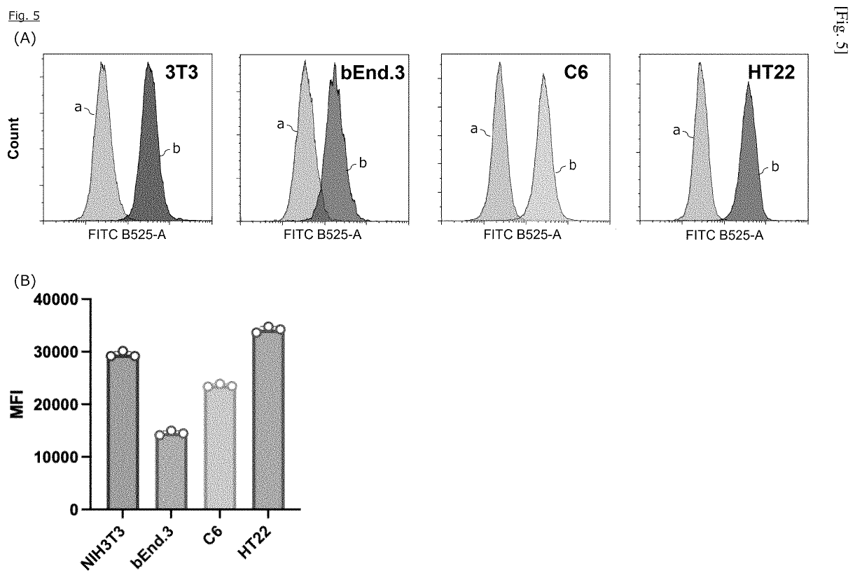

Fluorescence intensity in individual cells taken up the FITC-Insulin and in untreated cells (negative control) was measured by a flow cytometer for the NIH3T3 cells, bEnd.3 cells, C6 cells, and HT22 cells. FIG. 5 shows results. In FIG. 5A, code a indicates a histogram of the untreated cells (negative control), and code b indicates a histogram of the cells taken up the FITC-Insulin. FIG. 5B shows means and standard deviations of the fluorescence intensities for each type of cells. Similar to the results observed by the confocal microscopy, the FITC signals inside the cells were stronger in the NIH3T3 cells, C6 cells, and HT22 cells, which showed higher expression level of the insulin receptor, while the FITC signals inside the cells were weaker in the bEnd.3 cells, which showed lower expression level of the insulin receptor.

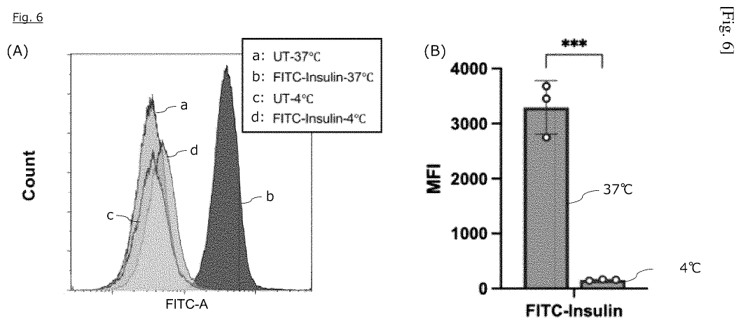

In FIG. 6, a fluorescence intensity when the FITC-Insulin was taken up by the HT22 cells at 37° C. and the HT22 cells at 4° C. were observed by the flow cytometry. FIG. 6 shows results. In FIG. 6A, code a indicates a histogram of the untreated cells (negative control) of the HT22 cells at 37° C., code b indicates a histogram when the FITC-Insulin was taken up by the HT22 cells at 37° C., code c indicates a histogram of the untreated cells (negative control) of the HT22 cells at 4° C., and code d indicates a histogram when the FITC-Insulin was taken up by the HT22 cells at 4° C. FIG. 6B shows means and standard deviations of the fluorescence intensities when the FITC-Insulin was taken up by the HT22 cells at 37° C. and means and standard deviations of the fluorescence intensities when the FITC-Insulin was taken up by the HT22 cells at 4° C. The amount of uptake of the FITC-Insulin at 37° C. was significantly higher than that at 4° C., suggesting that the intracellular uptake of the insulin is mediated by endocytosis in an insulin receptor-dependent manner.

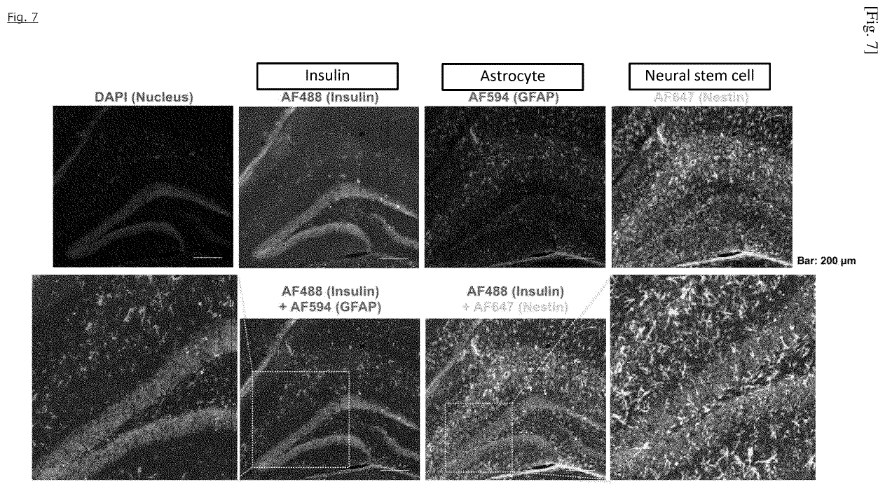

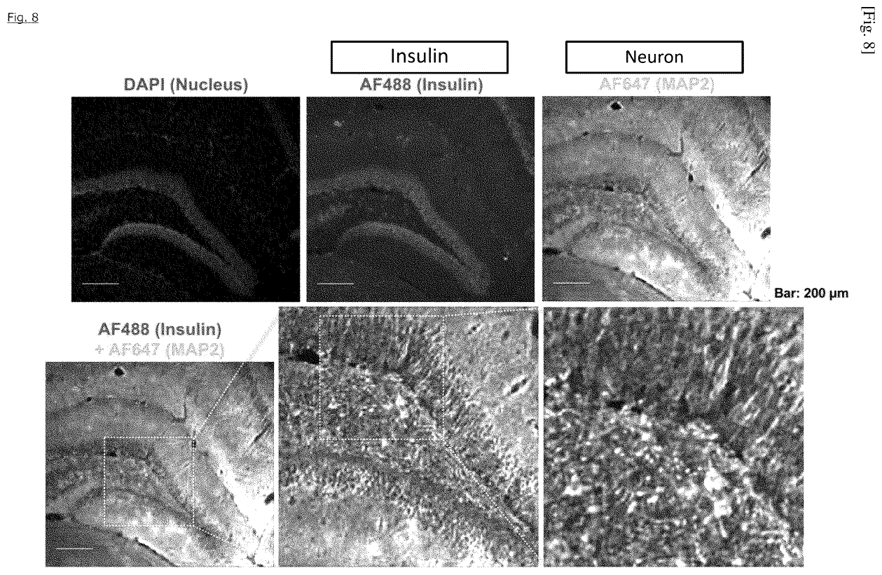

FIGS. 7 and 8 show images in which accumulation of the insulin in the hippocampus is observed when the insulin is administered nasally. In FIG. 7, the insulin is stained green with AF488. Glial fibrillary acidic protein (GFAP), which is a marker of astrocyte, is stained red with Alexa Fluor® 594 (AF594). Nestin, which is a marker of neural stem cell, is stained magenta with Alexa Fluor® 647 (AF647). The insulin delivered to the brain was also delivered to some astrocytes and neural stem cells, but the majority of the insulin was accumulated in other areas. In FIG. 8, the insulin is stained green with the AF488. Microtubule-associated protein 2 (MAP2), which is a marker of neuron, is stained magenta. The insulin colocalized with neurons (cell bodies) in the dentate gyrus. Thus, it was considered that the insulin could be a delivery carrier of a drug to the neuron.

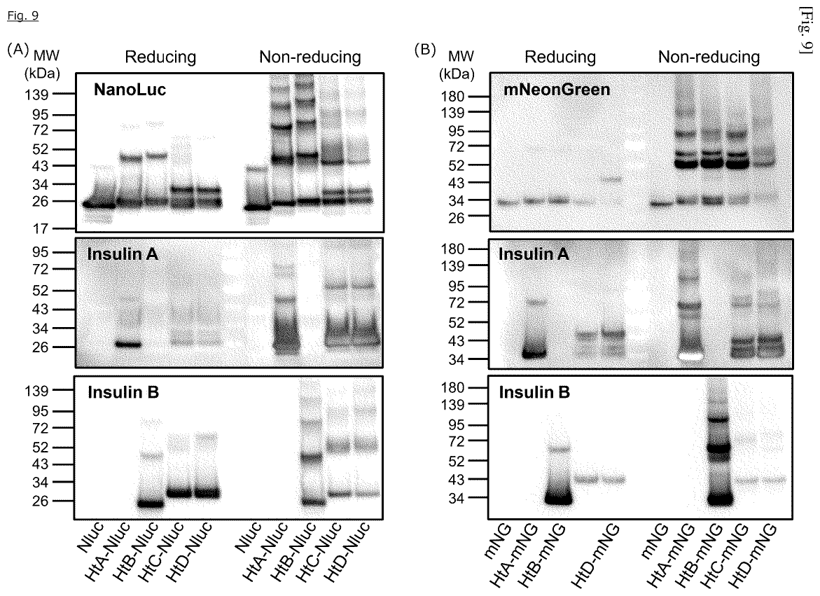

FIG. 9 shows results of expression analysis of the insulin fragment-fused proteins produced by Escherichia coli transfected with each of the constructs shown in FIG. 1. FIG. 9A shows results of a Western blot to identify a protein purified from the Escherichia coli expressing a protein obtained by fusing an insulin fragment to NanoLuc (Nluc). Upper panel shows a result of the Western blot using an antibody recognizing the Nluc, middle panel shows a result of the Western blot using an antibody recognizing insulin A-chain, and lower panel shows a result of the Western blot using an antibody recognizing insulin B-chain. FIG. 9B shows results of a Western blot to identify a protein purified from Escherichia coli expressing a protein obtained by fusing an insulin fragment to mNeonGreen (mNG). Upper panel shows a result of the Western blot using an antibody recognizing the mNeonGreen, middle panel shows a result of the Western blot using an antibody recognizing the insulin A-chain, and lower panel shows a result of the Western blot using an antibody recognizing the insulin B-chain. In each construct, a band of the peptide to be expressed could be identified.

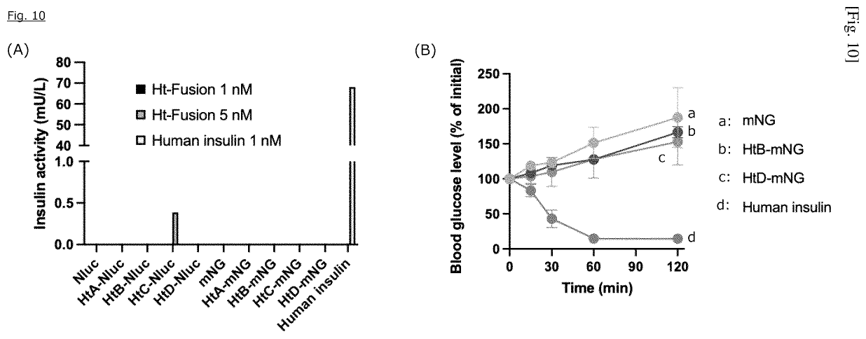

FIG. 10A shows insulin activities of proteins expressed from each construct. Slight insulin activity was shown when HtC-NLuc was used at 5 nM, but the insulin fragment-fused protein purified from each of the other constructs did not show insulin activity. FIG. 10B shows blood glucose levels in mice administered the mNG, HtB-mNG, HtD-mNG, and human insulin. The HtB-mNG and HtD-mNG, which are insulin fragment-fused proteins, did not show any activity to reduce the blood glucose levels.

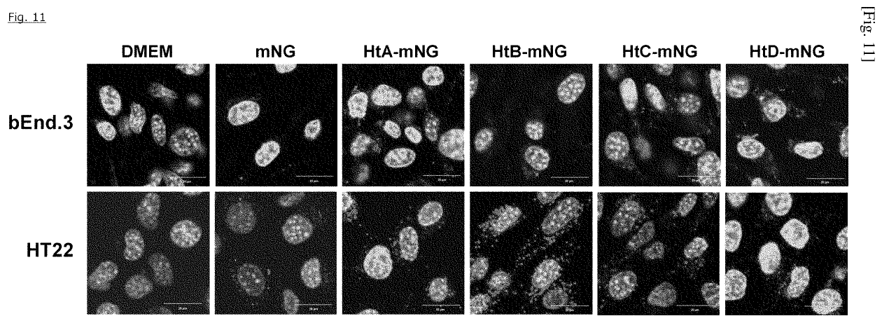

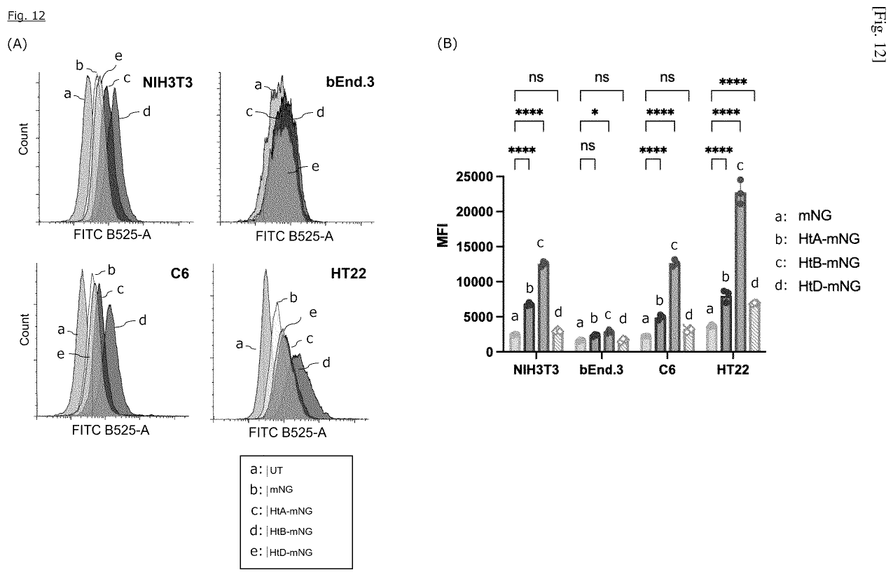

FIG. 11 shows confocal microscopy images showing uptake of insulin fragment-fused mNeonGreen when added to cultured cells. The insulin fragment-fused mNeonGreen was found in cytoplasm. FIG. 12A shows the fluorescence intensity of each type of cells shown in FIG. 11, analyzed by the flow cytometry. FIG. 12B shows a graph quantifying the results of FIG. 12A. In all cases of the insulin fragment-fused mNeonGreen (mNG), stronger fluorescence signals were found than in the case of the mNG. This indicates that the insulin fragment-fused protein is more efficient in intracellular uptake than a protein that is not fused with the insulin fragment.

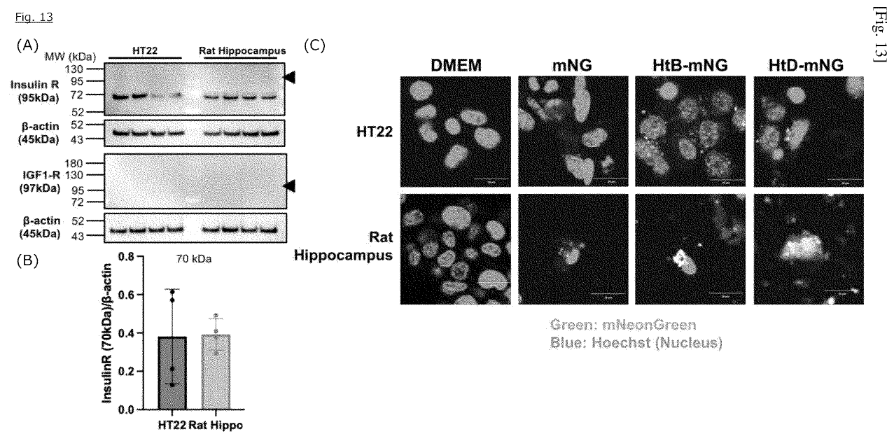

FIG. 13 shows results of experiments using the primary cultured rat hippocampal neurons. FIG. 13A shows results of expression of the insulin receptors in the primary cultured rat hippocampal neurons as observed by the Western blot. FIG. 13B shows a graph quantifying the results of FIG. 13A. The expression of the insulin receptor was founded in the primary cultured rat hippocampal neurons as well as in the HT22 cells used as a positive control. FIG. 13C shows confocal microscopy images when the insulin fragment-fused mNeonGreen was added to the cultured cells. An increased uptake of insulin fragment fusion protein could be observed in the primary cultured rat hippocampal neuron, which is similar to a native hippocampal neuron in vivo.

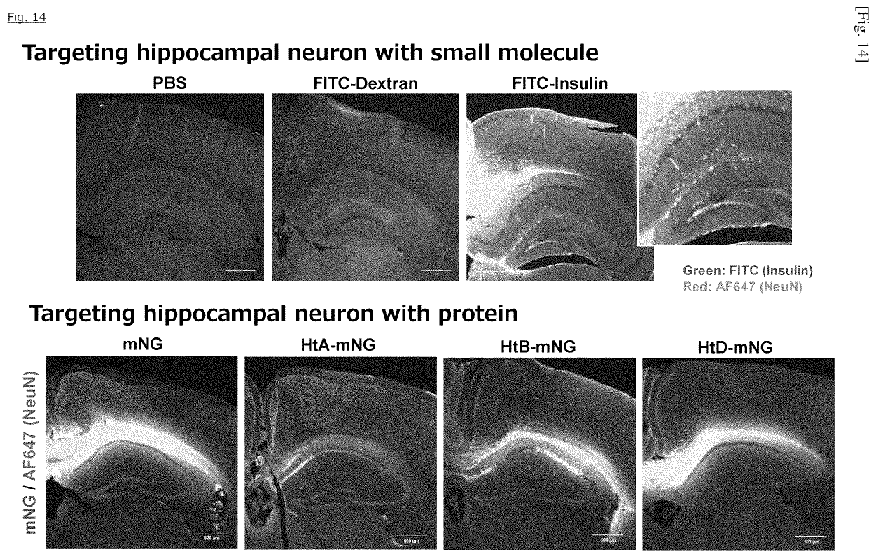

FIG. 14 shows results when the insulin fragment-fused mNeonGreen was administered nasally to a mouse. It was shown that both the HtA-mNG and HtC-mNG, which are the insulin fragment-fused mNeonGreen, were taken up into the neurons of the hippocampus.

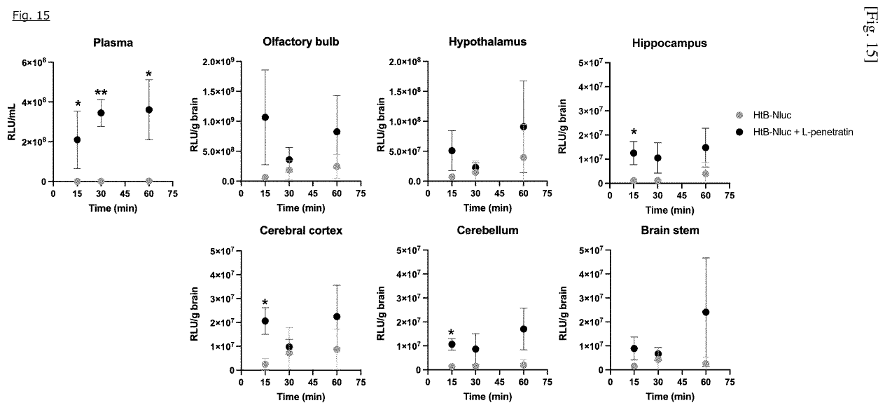

FIG. 15 shows a distribution of the HtC-mNG in plasma and in each brain tissue when the HtC-mNG was administered nasally. The distribution of the HtC-mNG was not limited to the hippocampus, but was also observed in an olfactory bulb, hypothalamus, cerebral cortex, cerebellum, and brainstem. This suggests that the insulin fragment-fused protein may be delivered not only to the hippocampus but also to the neurons in the brain that express the insulin receptor.

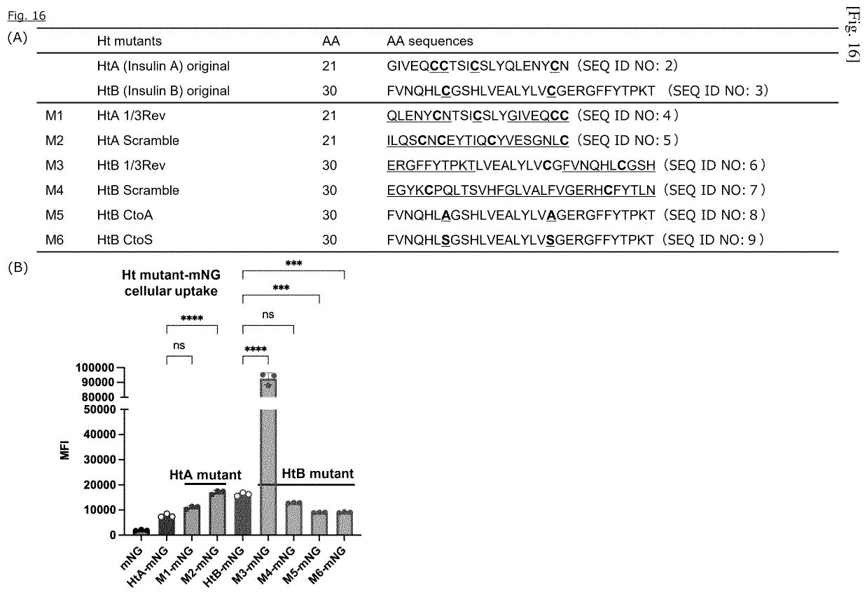

Next, delivery capacity of a variant of the insulin was examined. Each insulin fragment-fused mNeonGreen in which a peptide having one of the amino acid sequences M1 to M6 shown in FIG. 16A was fused to the mNeonGreen, was prepared and added to a culture medium for the HT22, C6, bEnd.3, and NIH3T3 cells, and the uptake thereof into the cells was evaluated by a mean fluorescence intensity measured by the flow cytometry. FIG. 16B shows results. The results showed that the insulin mutant was also effective in delivery to the cells.

Next, the extracellular vesicle containing the insulin fragment-fused mNeonGreen was produced to determine whether the insulin fragment could be used to deliver a lipid microparticle that serve as a carrier for the interested molecule such as a nucleic acid. The nucleic acid is delivered to the target tissue in a state where it is incorporated into the extracellular vesicle.

Cells expressing a construct for expressing a fusion protein of a CD63 to include the mNG inside the extracellular vesicle and fluorescently label it, and the mNeonGreen (CD63-mNG), and a construct for expressing a fusion protein of PTGFRN to present the insulin on the surface of the extracellular vesicle, insulin fragments (HtA, HtB, and HtC, respectively), and the mNeonGreen were prepared. For the CD63-mNG, the insulin fragment was introduced into an outer domain of cell membrane (extracellular domain). Similarly, HEK293T cells expressing the construct for expressing the fusion protein of the CD63 and mNeonGreen (CD63-mNG) and a construct for expressing a fusion protein of Lamp2b, an insulin fragment (Each of HtA, HtB, and HtC) for presenting insulin on the surface of extracellular vesicles, and the mNeonGreen were prepared. Culture supernatants were collected from each cell and extracellular vesicles were obtained by the size exclusion chromatography. Here, the CD63-mNG was used to increase the sensitivity of fluorescence observation. The PTGFRN and Lamp2b are proteins contained in a lipid bilayer structure of the extracellular vesicles.

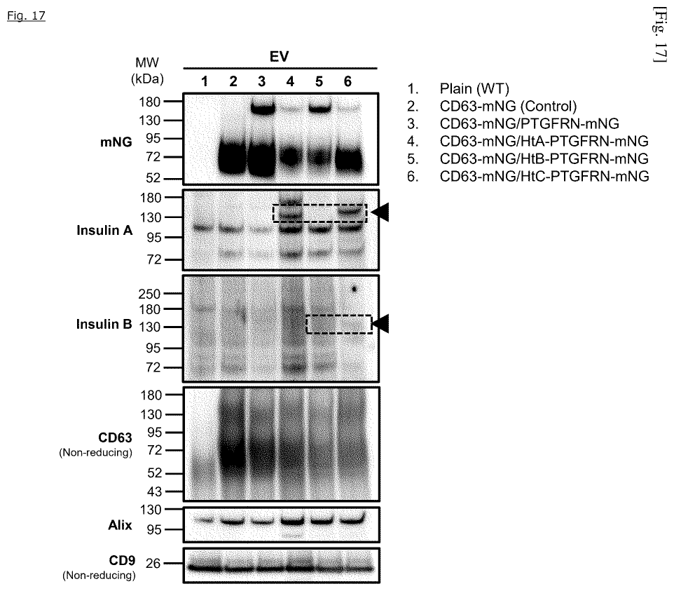

FIG. 17 shows results of the Western blot for the extracellular vesicles when the construct in which the insulin fragment was added to the PTGFRN was expressed. In the figure, “mNG” is a result of using a primary antibody against the mNeonGreen, “Insulin A” is a result of using a primary antibody against the insulin A-chain, “Insulin B” is a result of using a primary antibody against the insulin B-chain, “CD63” is a result of using a primary antibody against the extracellular vesicle protein CD63, “Alix” is a result of using a primary antibody against tan extracellular vesicle protein Alix, and “CD9” is a result of using a primary antibody against an extracellular vesicle protein CD9.

By adding the insulin fragment to the PTGFRN, the insulin fragment could be added to the extracellular vesicle.

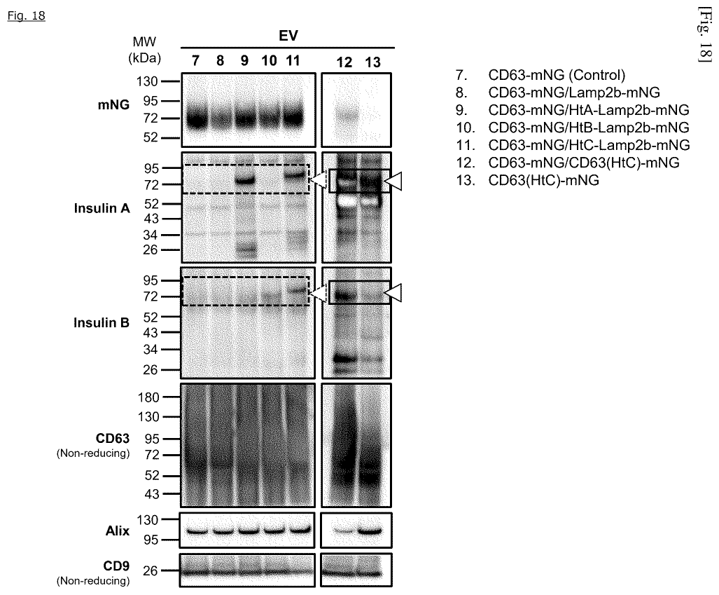

FIG. 18 shows results of the Western blot for the extracellular vesicles when the construct in which the insulin fragment was added to the Lamp2b was expressed. In the figure, “mNG” is a result of using a primary antibody against the mNeonGreen, “Insulin A” is a result of using the primary antibody against the insulin A-chain, “Insulin B” is a result of using the primary antibody against the insulin B-chain, “CD63” is a result of using the primary antibody against the extracellular vesicle protein CD63, “Alix” is a result of using the primary antibody against the extracellular vesicle protein Alix, and “CD9” is a result of using the primary antibody against the extracellular vesicle protein CD9.

By adding the insulin fragment to the Lamp2b, the insulin fragment could be added to the extracellular vesicle.

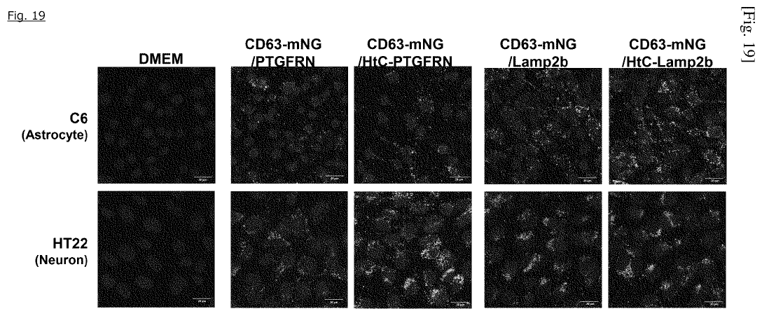

Subsequently, the extracellular vesicles prepared above were added to culture supernatants of the C6 and HT22 cells, and localization of fluorescence in these cells was observed. FIG. 19 shows results. In both cases where the mNG was added to the PTGFRN and the mNG was added to the Lamp2b, accumulation of fluorescent signals in a cytoplasm was greater in the case where the HtC was added.

These results indicate that addition of insulin fragments allows efficient delivery of the contents of the extracellular vesicles to the target.

### SEQUENCE LISTING

P24-053WO_PCT_insulin fragment_20240308_163839_0.xml

1. An insulin fragment tag for delivering interested molecules to neurons,
the insulin fragment tag comprising a polypeptide including an amino acid sequence that has at least 90% identity with the amino acid sequence of at least one chain selected from an insulin A-chain and an insulin B-chain, and the insulin fragment tag having binding capacity insulin receptors.

2. The insulin fragment tag according to claim 1, wherein the insulin fragment tag has no activity as insulin.

3. A pharmaceutical composition for treating or preventing a nervous system disease, containing an interested molecule to which the insulin fragment tag added according to claim 1.

4. The pharmaceutical composition according to claim 3, containing a pharmacologically active component in lipid microparticles having the insulin fragment tag on the surface.

5. A polynucleotide encoding the insulin fragment tag according to claim 1.

6. A vector containing the polynucleotide according to claim 5.

7. A cell into which the vector is introduced according to claim 6.

8. A polynucleotide encoding the insulin fragment tag according to claim 1 and a peptide or polypeptide that is an interested molecule.

9. A vector containing the polynucleotide according to claim 8.

10. A cell into which the vector according to claim 9 is introduced.


SEQUENCE LISTING (9 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, synthetic construct
GIVEQCCTSICSLYQLENYCN

SEQ ID NO: 3 - protein, 30 aa, synthetic construct
FVNQHLCGSHLVEALYLVCGERGFFYTPKT

SEQ ID NO: 4 - protein, 21 aa, synthetic construct
QLENYCNTSICSLYGIVEQCC

SEQ ID NO: 5 - protein, 21 aa, synthetic construct
ILQSCNCEYTIQCYVESGNLC

SEQ ID NO: 6 - protein, 30 aa, synthetic construct
ERGFFYTPKTLVEALYLVCGFVNQHLCGSH

SEQ ID NO: 7 - protein, 30 aa, synthetic construct
EGYKCPQLTSVHFGLVALFVGERHCFYTLN

SEQ ID NO: 8 - protein, 30 aa, synthetic construct
FVNQHLAGSHLVEALYLVAGERGFFYTPKT

SEQ ID NO: 9 - protein, 30 aa, synthetic construct
FVNQHLSGSHLVEALYLVSGERGFFYTPKT